(19) United States
(12) Patent Application Publication
(10) Pub. No.: US 2025/0285757 A1
(43) Pub. Date: Sep. 11, 2025

(54) SYSTEM FOR AND METHOD OF ZINC IMAGING FOR MARGIN ASSESSMENT DURING BREAST CONSERVING SURGERY

(71) Applicant: The General Hospital Corporation, Boston, MA (US)

(72) Inventors: Maria Veronica Clavijo Jordan, Charlsetown, MA (US); Clarissa Z. Cooley, Charlestown, MA (US); Yuwen I. Zhou, Charlestown, MA (US)

(21) Appl. No.: 18/861,837

(22) PCT Filed: May 1, 2023
(86) PCT No.: PCT/US2023/066454

§ 371 Date: Oct. 30, 2024

Related U.S. Application Data
(60) Provisional application No. 63/337,087, filed on Apr. 30, 2022.

(51) Int. Cl. G16H 50/20

(52) U.S. Cl. CPC G16H 50/20

(57) ABSTRACT

Methods of detecting and measuring positron emission for tumor margin assessment are described. The preferential uptake of radiotracers by certain tumor types provides a measurable parameter for distinguishing between healthy and cancerous tissue to enhance tumor margin determination. The methods provide more accurate margin delineation and reduced tissue resection size relative to conventional breast conserving surgery techniques.

### CROSS REFERENCE TO RELATED APPLICATIONS

The present application claims priority to U.S. Provisional Patent Application No. 63/337,087, filed Apr. 30, 2022. The application is herein incorporated by reference in its entirety.

### BACKGROUND

The treatment for breast cancer has evolved over time: from radical mastectomy to more localized resection for breast conserving surgery (BCS), which is less disfiguring and has fewer morbidities. Due to the lack of specific intra-operative imaging tools to effectively delineate malignant margins, tumor resection size is either aggressively conservative and thus results in unsatisfactory cosmetic results, or ˜20-40% of tumors removed have a positive margin of cancer cells requiring patients to return for surgical re-excision.

Radioguided surgery (RGS) is technique used to increase the precision of tumor resection. The technique involves administering a radiotracer to a patient, which is preferentially taken up by a tumor due to the upregulation of import transporters specific to the radiotracer. A detector sensitive to radiation emitted by the tracer is used to identify areas of high radiotracer uptake, and thereby delineate the tumor margin. Current methods use gamma (γ) emitting tracers and γ-radiation detectors. However, γ radiation penetrates deep into tissues and may be taken up by healthy tissue and nearby organs and thereby contribute non-negligible background signal.

### SUMMARY

It is an aspect of the present disclosure to provide a method for breast tumor margin assessment. The method includes administering a radiotracer containing a radioisotope of zinc to a patient. A positron detection probe is positioned proximate a region-of-interest in a breast of the patient. Positron emission data are acquired from the region-of-interest using the positron detection probe. The positron emission data are received with a computer system. A tumor margin in the region-of-interest is determined from the positron emission data using the computer system. A report indicating the determined tumor margin is generated by the computer system, and may be displayed to a user.

It is another aspect of the present disclosure to provide a method of breast tumor margin assessment that includes receiving positron emission data with a computer system, where the positron emission data include measurements of positron emissions from breast tissues in a region-of-interest that have taken up a previously administered radiotracer containing a zinc radioisotope. A tumor margin map that depicts a spatial distribution of the positron emissions in the region-of-interest is generated from the positron emission data. The tumor margin map may be displayed with the computer system.

### BRIEF DESCRIPTION OF THE DRAWINGS

FIG. 1 is a non-limiting example of a system for measuring positron emission, according to aspects of the present disclosure.

FIG. 2 is a non-limiting example of a method for determining tumor margins using a positron detection system such as the system in FIG. 1, according to aspects of the present disclosure.

FIG. 3 is a non-limiting example of a method of measuring positron emission using a positron detection system such as the system in FIG. 1, according to aspects of the present disclosure.

FIG. 4 is a schematic of the progressive increase in zinc uptake and concentration ([Zn]) of mammary gland epithelium from normal cells to progressively more aggressive cancer cells.

FIG. 5A shows the preferential uptake of zinc by breast cancer cells in hematoxylin and eosin (H&E) stained sections of mammary gland tissue with numbered regions of cancerous tissue.

FIG. 5B shows the preferential uptake of zinc by breast cancer cells in an X-ray fluorescence image (XRF) image of the same sectioned mammary gland tissue of FIG. 5A, illustrating increased [Zn] concentrations in the cancerous tissue compared to adjacent healthy tissue.

FIG. 6 is a block diagram of an example system for tumor margin assessment, such as breast tumor margin assessment, according to some examples described in the present disclosure.

FIG. 7 is a block diagram of example components that can implement the system of FIG. 6.

### DETAILED DESCRIPTION

Methods of detecting and measuring positron emission for tumor margin assessment using a positron emitting zinc radiotracer. The preferential uptake of zinc radiotracers by certain tumor types provides a measurable parameter for distinguishing between healthy and cancerous tissue to enhance tumor margin determination. The methods provide more accurate margin delineation and reduced tissue resection size, for instance as relative to conventional breast conserving surgery (BCS) techniques.

To address the problem of positive tumor margin following a tumor resection (e.g., BCS), the disclosed methods take advantage of the specific upregulation of zinc import transporters and subsequent zinc accumulation in malignant breast cancer and not in healthy breast stroma. Breast tumor margin may be assessed by detecting positron emissions (e.g., β+ radiation) using a positron emitting radiotracers and suitable positron emission detector. β− radiation, including positron emission, has a short range in tissues (e.g., ˜1 mm), thus allowing for the detection of superficial radiation. Positron detection probes are sensitive to positron emissions over a short range of radiation, thus avoiding the problem of background γ-radiation without the need for lateral and back shielding, while being more sensitive and accurate in localizing tumoral lesions than conventional γ-detector-based imaging (e.g., positron emission tomography (PET)).

In breast tissue, zinc plays an important role in normal mammary gland growth, normal mammary gland development, and post-lactation transformations, some of which may occur during lactation. Breast cancer tissues show significantly higher zinc levels compared with normal breast tissue. Furthermore, zinc distribution and zinc transporter levels show distinct profiles in breast cancer subtypes. For example, luminal breast cancer tumors express higher levels of the ZIP6 gene compared to basal and HER2 overexpression tumors, and ZIP8 gene expression is greater in high-grade versus low-grade ER+ tumors. Further, higher gene expression of ZIP10 is observed in basal cells and higher gene expression of ZIP6 in luminal cells. At the protein level, basal cells display higher levels of ZnT1, ZIP1, and ZIP10 compared to normal cells; and luminal cells display higher levels of all ZnT proteins ZIP3, ZIP5, ZIP6, ZIP8, and ZIP10. The differing zinc transporter profiles and their effects on cellular zinc homeostasis may contribute to the phenotypic differences (including invasiveness, metastasis, and treatment effectiveness) in breast cancer subtypes. Specifically, ZIP10 and ZIP6 have been linked to tumor migration in both basal and luminal breast cancers. In basal breast cancer cells, ZIP10 functions as a zinc importer and ZIP10-mediated zinc uptake stimulates cell migration.

In another example, the zinc importers ZIP1, ZIP10, and ZIP14 are expressed on the plasma membrane of pancreatic a cells. ZIP4 is expressed on the plasma membrane of pancreatic β cells, which produce a zinc: insulin complex stored in zinc-rich secretory glanules, suggesting that ZIP4 contributes to maintaining adequate zinc levels in the β cells for insulin secretion. Dysregulation of cellular zinc homeostasis has been implicated in the progression of pancreatic cancer.

In another example, the human prostate gland contains exceptionally high levels of zinc (˜800-1500 μM in prostate epithelial cells compared to ˜100-500 μM in other soft tissue cells), and the zinc concentration in prostatic fluid is about 500-fold greater than in plasma. Zinc accumulation in prostate epithelial cells is attributed to the expression of ZIP1, which is present on the basolateral membrane and functions as the major importer of zinc from circulating blood plasma into prostate cells. In addition to ZIP1, ZIP2, and ZIP3 have been identified on the apical surface of normal prostate cells, and it is speculated that these proteins function in the reuptake of zinc from the prostatic fluid. Compared with normal or nonmalignant prostate tissue, malignant prostate tissue displays markedly lower levels of zinc, and the decline in zinc occurs early in the malignant transformation. Concomitant with the declining zinc levels, ZIP1 gene expression is downregulated in prostate cancer. Other ZIP family members, ZIP2, ZIP3, and ZIP4, also show reduced expression in prostate cancer cells.

Thus, the change in intracellular zinc concentration acts as an indicator of healthy or cancerous tissue. In a non-limiting example of the present disclosure, a zinc radiotracer is used to assess tumor margin by imaging tissues in the margin using a positron detection probe or detector. The positrons emitted by zinc radiotracers travel a short path inside the patient's body before annihilating with an electron in the tissue. This superficial radiation can be measured using a positron detection probe to assess tumor margin in local tissue regions, providing an accurate and spatially localized measurement of radiotracer uptake.

The zinc radiotracer may include a positron emitting zinc cation. A positron emitting zinc cation may include any radioactive isotopes of zinc. In some examples, the positron emitting zinc cation may be ⁶²Zn, ⁶³Zn, ⁶⁵Zn, or another suitable radioisotope of zinc. As a potential radioisotope, ⁶³Zn possesses a half-life of 38.5 minutes, meaning the radioisotope may be well-suited for kinetic studies with good signal-to-noise characteristics over a period of 2-3 hours following administration. The zinc cation may be reacted with an anion to produce a positron emitting zinc compound as the radiotracer. As one non-limiting example, the anion may be citrate. In these instances, the radiotracer may be radiolabeled zinc citrate, such as ⁶³Zn zinc citrate. Other molecules or compounds containing a radioisotope of zinc may also be used as zinc radiotracers. For example, a zinc radioisotope may be conjugated with a targeting antibody, or used to radiolabel other molecules or compounds.

Advantageously, zinc radiotracers may be used for intraoperative detection of cancer cells for breast cancer, pancreatic cancer, prostate cancer, or other cancers in which the zinc transporters are upregulated compared to healthy tissue; that is, for cancerous tissues where there is a preferential uptake of zinc radiotracer as compared to healthy tissue. Accordingly, the zinc radiotracers can be used to assess tumor margin by measuring zinc radiotracer activity in tissues, such as cancerous breast tissue. Because positrons have a short range in tissues (e.g., on the order of 1 mm), a positron detection probe can be used to directly measure the superficial positron emission in cancerous breast tissues, thereby providing for an assessment of tumor margin following a tumor resection. For instance, as will be described, the positron detection probe can be used to detect and measure superficial positron emissions from cancerous cells not removed in the tumor resection. By mapping the locations of these superficial positron emissions, the tumor margin remaining after resection can be determined.

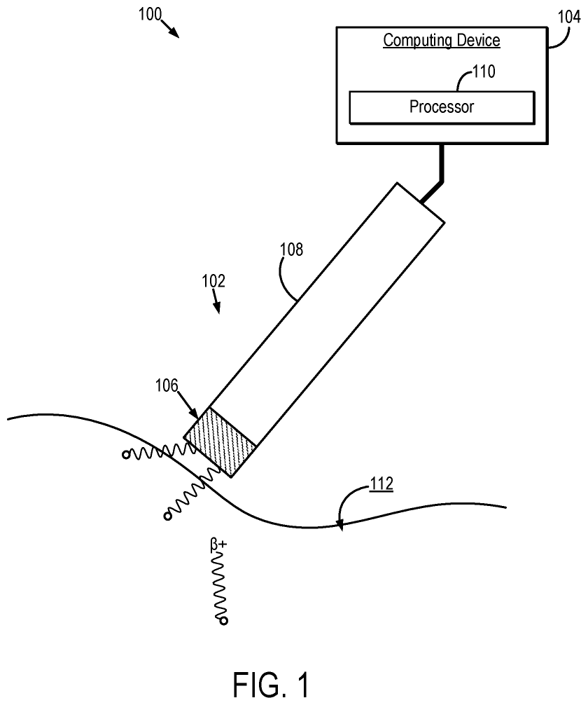

Referring now to FIG. 1, a tumor margin assessment system 100 is shown. The tumor margin assessment system 100 generally includes a positron detection probe 102 that is in communication with a computing device 104. The positron detection probe 102 generally includes a radiation detector 106 coupled to, or otherwise housed within, a housing 108.

In general, the radiation detector 106 is sensitive to a short range of positron emission radiation. The radiation detector 106 may be a gaseous ionization detector, a scintillation detector, a semiconductor detector, or the like. For example, the radiation detector 106 may include a scintillator, a phoswich detector, a surface barrier detector, an ion-implanted silicon detector, and so on.

The positron detection probe 102 may be in communication with the computing device 104 via a wired connection, a wireless connection, or both. For example, the positron detection probe 102 may be connected to the computing device 104 via a wired connection. The wired connection may allow for the transfer of data (e.g., positron detection counts) from the positron detection probe 102 to the computing device 104. The wired connection may also provide power to the positron detection probe 102. In other examples, the positron detection probe 102 may be in communication with the computing device 104 via a wireless connection, such as an optical data transfer, or the like. In these instances, the positron detector probe 102 may be powered via a separate power cable, an internal battery, or the like.

The computing device 104 may include a processor 110 that is configured to control operation of the positron detection probe 102 and/or to receive and processor data measured by the radiation detector 106. In some examples, the positron detector probe 102 may also have radiation detection electronics housed within the housing 108. For instance, a pre-amplifier for amplifying and shaping the electrical signal output from the radiation detector 106, an amplifier for amplifying the electrical signal output from the radiation detector 106, and/or a discriminator for filtering noise and/or reducing gamma ray components of the electrical signal output by the radiation detector 106 may all be housed within the housing 108. Additionally or alternatively, the amplifier and discriminator may be remote from the housing 108. For instance, the computing device 104 may include an amplifier and/or discriminator. As noted above, the computing device 104 may include a processor 110 and/or other detection electronics. In some examples, the computing device 104 may receive the electrical signal output from the radiation detector 106 and function as a counter by processing the electrical signal to count the positrons detected by the radiation detector 106. Accordingly, the computing device 104 may receive electrical signals from the positron detector probe 102 and may process the electrical signals to generate and store positron emission data, which may include positron counts, measured activity, or the like.

The housing 108 may be sized and shaped to allow for a handheld operation of the positron detection probe 102. In some implementations, the positron detection probe 102 is configured for intraoperative use, such that the positron detection probe 102 may be introduced into the internal anatomy of a patient. For example, the positron detection probe 102 may be configured to be intraoperatively positioned within a resection cavity, or the like. In this way, the positron detection probe 102 may be placed into direct contact, or near direct contact, with a tissue 112. As a non-limiting example, the tissue 112 may include breast tissue within a breast tumor resection cavity. In some other implementations, the positron detection probe 102 may be configured to be placed into contact, or near contact, with the skin surface of a patient.

One or more position trackers may be coupled to the housing 108 in some examples. The position trackers can be used to track the spatial location (e.g., position and orientation) of the positron detection probe 102 relative to a frame of reference (e.g., a reference coordinate system, a co-registered medical image of the patient's anatomy). By tracking the position of the positron detection probe 102, the measured positron emissions can be accurately mapped to generate a position emission map over the scanned tissue region (e.g., the resection margin). In this way, the measured positron emission activity can be mapped to the tissue anatomy to identify regions of tumor margin that need further resection. As an example, position trackers may include magnetic trackers, optical trackers, radio frequency trackers, ultrasound trackers, infrared trackers, or the like

In some examples, the housing 108 may be coupled to a surgical robot for robot-assisted operation of the positron detection probe 102. For instance, the housing 108 may be coupled to a robotic arm, which can maneuver the positron detection probe 102 into position to detect positron emissions from tissues in the patient that have an uptake of radiotracer, such as the zinc radiotracers described in the present disclosure. In this way, the positron detection probe 102 can be positioned into place by the same surgical robot system that may also be used to perform a tumor resection procedure.

In still other examples, the housing 108 may be coupled to an endoscope, such as a flexible endoscope. In these instances, the positron detection probe 102 may be introduced into a resection cavity or other internal cavity of the patient by maneuvering the endoscope.

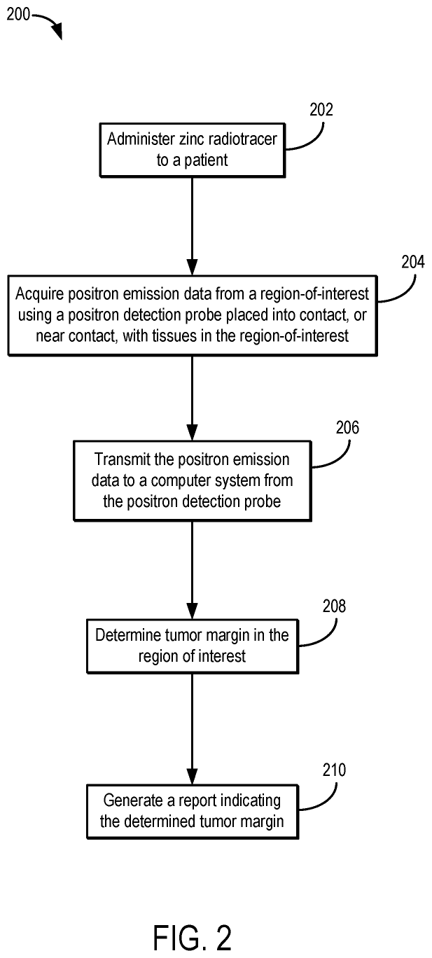

Referring now to FIG. 2, an example method 200 of assessing breast tumor margin using a zinc radiotracer is shown. In step 202, a zinc radiotracer is administered to a patient. As described above, in some examples, the radiotracer may a radiotracer containing a radioisotope of zinc, such as ⁶²Zn, ⁶³Zn, or ⁶⁵Zn. As a non-limiting example, the radioisotope may be ⁶³Zn and the radiotracer may be radiolabeled zinc citrate; that is, ⁶³Zn-zinc citrate. Other zinc-containing radiotracers may also be used. The radiotracer may be injected intravenously. Alternatively, the radiotracer may be administered by inhalation, oral ingestion, or by direct injection into an organ or tissue. The radiotracer circulates and is taken up by the body. As one non-limiting example, the radiotracer may be administered 20-30 min before measuring positron emissions, which in some examples may include 20-30 minutes before performing a tumor resection procedure.

At step 204, a positron detection probe, such as the one shown in FIG. 1, is placed in proximity to a region-of-interest and positron emission data are acquired by measuring positron emissions from tissues within the region-of-interest using the positron detection probe. That is, the positron detection probe may be placed into contact, or near contact, with one or more tissues in the region-of-interest to measure positron emission (e.g., superficial positron emissions) from any tissues in the region-of-interest that have preferentially taken up the zinc radiotracer. In a non-limiting example, the region-of-interest may be the breast of a patient, or a region within the breast of a patient (e.g., a lumpectomy cavity or other tumor resection cavity). The positron detection probe detects the positron emissions from the radiotracer based on varying accumulation in tissues in the region-of-interest. The positron emission data may include positron counts, measurements of radioisotope (i.e., zinc radioisotope) activity, measurements of radioisotope (e.g., zinc radioisotope) activity concentration, or combinations thereof.

The positron emission data are transmitted, communicated, or otherwise transferred to a computer system (e.g., computing device 104) at step 206. As described above, the positron emission data may be communicated to the computer system via a wired connection, a wireless connection, or combinations thereof.

Once the positron emission data have been transmitted, the computer system determines a tumor margin in the region-of-interest at step 208. The tumor margin may be determined by analyzing the measured positron emissions from the tissue in the region-of-interest. Tissues that have a higher level of measured positron emissions are indicative of cancerous tissues in the tumor margin. As a non-limiting example, the positron emission data may be compared to a threshold, such that measurements of positron emissions above the threshold indicate the presence of cancerous tissue in the tumor margin. A threshold level may be set by a user to delineate zinc concentrations in healthy tissue from the increased zinc concentrations in cancerous tissues. The threshold level may be adjusted based on cancer type.

At step 210, a report is generated by the computer system, which may indicate the determined tumor margin. In some examples, the report may include a tumor resection plan including the identification of healthy and cancerous tissues in the region-of-interest based on the measured positron emissions and determined tumor margin. The report may be generated on a display, such as a display of the computing device 104. In some examples, the report may include a tumor margin map that depicts the spatial distribution of positron emissions from tissues in the region-of-interest. In other examples, the report may include an overlay of measured positron emissions on other medical images acquired from the region-of-interest. For instance, an x-ray or other image of the breast of the subject may be displayed to a user and a heat map or other type of display element indicating the measured positron emissions in the determined tumor margin may be generated and overlaid on the medical image. In some examples, generating the report may include retrieving position data measured by one or more position trackers on the positron detection probe and spatially localizing the positron emissions relative to patient anatomy based on the position data. The position data may also be used to co-register one or more medical images with the position emission data and/or determined tumor margin. The report may be generated in real-time. In a non-limiting example, the cancerous tissue may be highlighted. Alternatively, healthy tissue and cancerous tissue may be highlighted in different colors, shadings, or patterns.

In a non-limiting example, for a breast tumor resection procedure the operating room (“OR”) workflow may include first administering a zinc radiotracer (e.g., ⁶³Zn-zinc citrate, or other ⁶³Zn containing compounds) to a patient a preset amount of time before the procedure. For example, the zinc radiotracer may be administered 20-30 minutes before entering the OR, which will allow for the radiotracer to be taken up by cancer cells via the upregulated zinc transporters. After the tumor is resected, a positron detection probe (e.g., a wireless handheld positron detection probe) may be used for intra-cavity examination of positive tumor margin. The positron detection probe is designed to fit into lumpectomy incisions as small as 2 inches. Positron emission data are acquired using the positron detection probe, and images of the area under screening are reconstructed and sent to the readout unit, which gives the surgeon real-time feedback about the margin status (e.g., positive or negative based on calibration and detection algorithms determined from benchtop and preclinical studies) and guide the surgeon in the removal of any leftover cancer tissues.

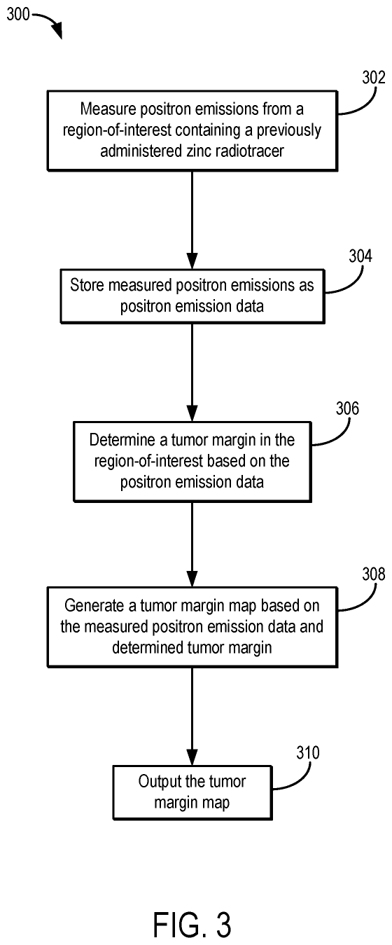

Referring now to FIG. 3, an example method 300 for measuring positron emissions and generating a positron emission map therefrom is shown. At step 302, positron emissions are detected by a positron detection probe in proximity with a region-of-interest. The positron detection probe detects the positron emissions from tissues in the region-of-interest that have taken up a previously administered zinc radiotracer. The measured positron emissions are stored as positron emission data, as indicated at step 304. As described above, the positron emission data may include positron counts, radioisotope activity, radioisotope activity concentration, or combinations thereof.

Next, as step 306, the varying levels of positron emissions measured in the positron emission data are analyzed by a computer system (e.g., computing device 104) to distinguish between healthy and cancerous tissue, based on preferential uptake of the previously administered zinc radiotracer (e.g., ⁶³Zn-zinc citrate). Accordingly, a tumor margin in the region-of-interest may be determined by analyzing the positron emission data. As previously described above with respect to FIG. 2, the healthy cells and cancerous cells may be distinguished from one another by their relative intracellular zinc concentrations, which may affect the positron emissions measured from the cells. A threshold [Zn] may be set to classify cells into healthy or cancerous cells. The threshold may also be adjusted based on the type of cancer. A tumor margin can thus be determined by delineating between healthy and cancerous tissue.

At step 308, images of the region-of-interest are generated by the computer system from the positron emission data. In some examples, the images may include indications of the healthy and cancerous tissue. In these instances, the images may be tumor margin maps that depict a spatial distribution of measured positron emissions indicative of a tumor margin. In a non-limiting example, the cancerous tissue may be highlighted in the tumor margin map. Alternatively, healthy tissue and cancerous tissue may be highlighted in different colors, shadings, or patterns.

The tumor margin map may then be output by the computer system, as indicated at step 310. For instance, the tumor margin map may be displayed to a user via a display of the computer system. In other instances, the tumor margin map may be displayed together with other images or data, such as other medical images of the patient and other relevant data. The tumor margin map may be stored as part of a report and the report displayed to a user. The report may indicate a tumor resection plan, such as by highlighting regions in the region-of-interest that are indicated as tumor margin that should be resected.

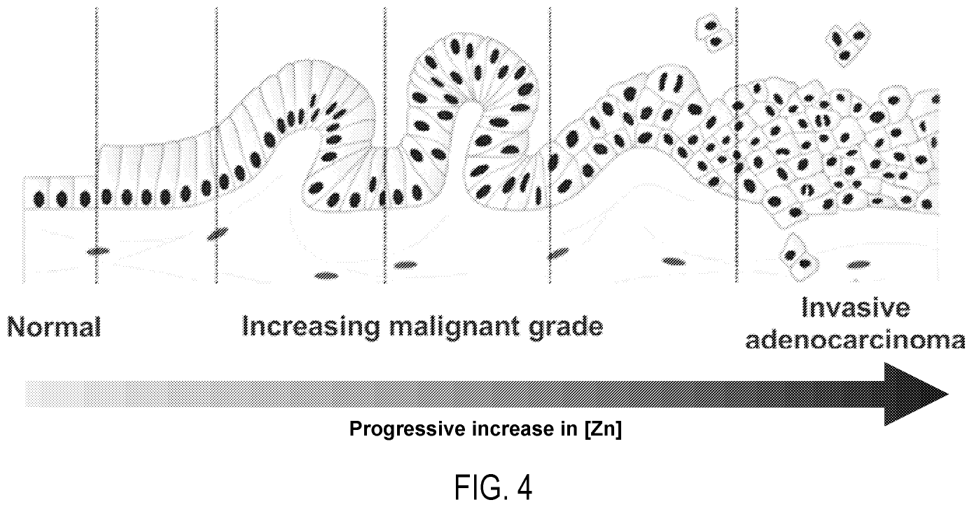

FIG. 4 shows a schematic of mammary gland tissue cellular morphology, ranging from normal (i.e., healthy) cells on the left side of the image, to increasingly malignant cancer cell morphologies. As previously described, intracellular [Zn] may vary between normal and cancerous cells due to the upregulation of zinc transporters in cancer cells. This results in a preferential uptake of zinc radiotracers in cancerous cells, such as cancerous breast tissue cells. Accordingly, the preferential uptake of zinc radiotracer into cancerous breast tissue cells allows for directly imaging the tumor margin remaining after a resection procedure.

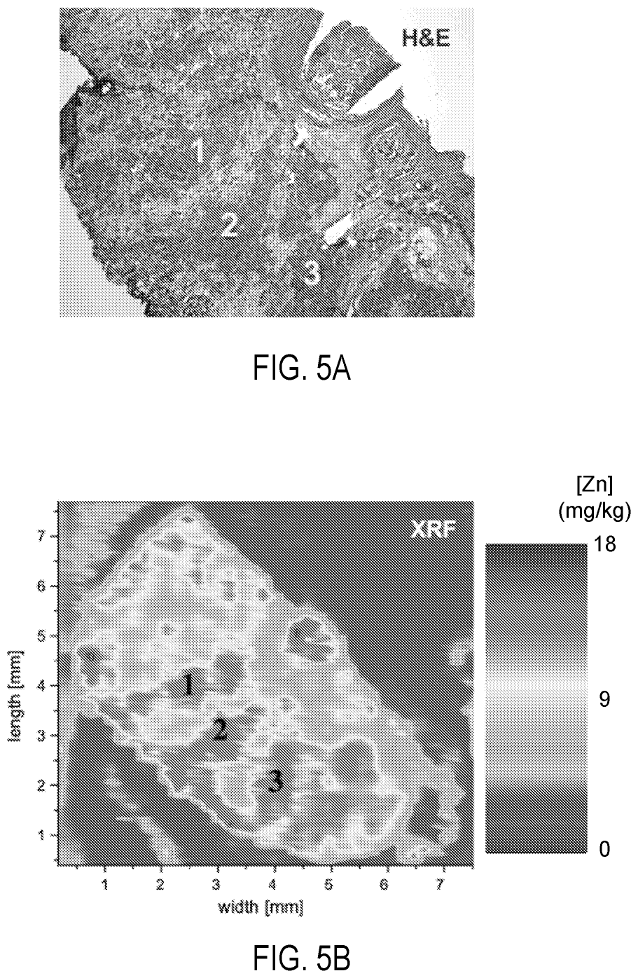

Next, FIGS. 5A-BB show a non-limiting example of the effects of the preferential uptake of a zinc radiotracer in a section mammary gland tissue sample. FIG. 5A illustrates the H&E staining, with identified regions of cancerous tissue (labeled 1, 2, 3). FIG. 5B shows a complementary XRF image of the sample indicating zinc concentration. The same labeled cancerous tissue regions indicate a higher concentration of intracellular Zn relative to adjacent healthy tissue.

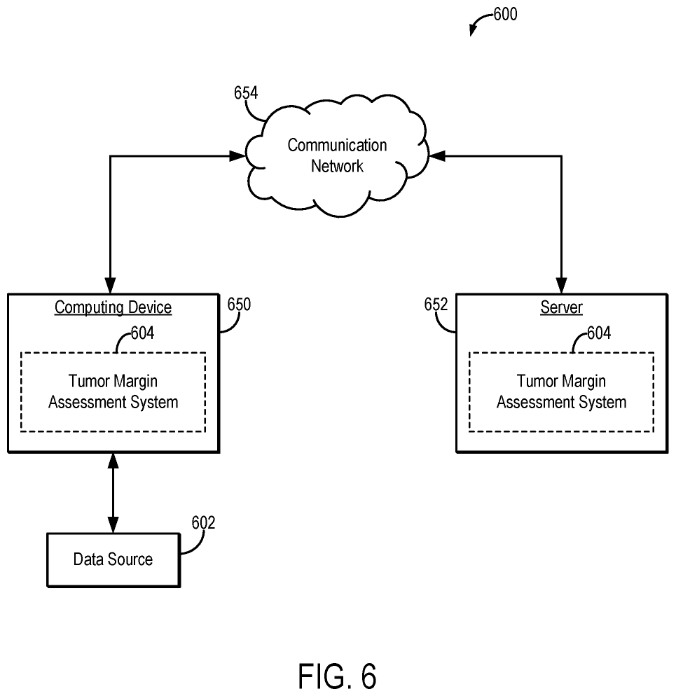

Referring now to FIG. 6, an example of a system 600 for tumor margin assessment in accordance with some embodiments of the systems and methods described in the present disclosure is shown. As shown in FIG. 6, a computing device 650 can receive one or more types of data (e.g., positron emission data, positron detection probe position data, medical image data) from data source 602. In some embodiments, computing device 650 can execute at least a portion of a tumor margin assessment system 604 to identify positive tumor margin in a region-of-interest from data received from the data source 602.

Additionally or alternatively, in some embodiments, the computing device 650 can communicate information about data received from the data source 602 to a server 652 over a communication network 654, which can execute at least a portion of the tumor margin assessment system 604. In such embodiments, the server 652 can return information to the computing device 650 (and/or any other suitable computing device) indicative of an output of the tumor margin assessment system 604.

In some embodiments, computing device 650 and/or server 652 can be any suitable computing device or combination of devices, such as a desktop computer, a laptop computer, a smartphone, a tablet computer, a wearable computer, a server computer, a virtual machine being executed by a physical computing device, and so on. The computing device 650 and/or server 652 can also reconstruct images from the data. The computing device 650 may be the same as computing device 104 in FIG. 1, or may be a separate computing device that is in communication with computing device 104 (e.g., via a wired and/or wireless connection).

In some embodiments, data source 602 can be any suitable source of data (e.g., measurement data, images reconstructed from measurement data, processed image data), such as a positron detection probe, another computing device (e.g., a server storing measurement data, images reconstructed from measurement data, processed image data), and so on. In some embodiments, data source 602 can be local to computing device 650. For example, data source 602 can be incorporated with computing device 650 (e.g., computing device 650 can be configured as part of a device for measuring, recording, estimating, acquiring, or otherwise collecting or storing data). As another example, data source 602 can be connected to computing device 650 by a cable, a direct wireless link, and so on. Additionally or alternatively, in some embodiments, data source 602 can be located locally and/or remotely from computing device 650, and can communicate data to computing device 650 (and/or server 652) via a communication network (e.g., communication network 654).

In some embodiments, communication network 654 can be any suitable communication network or combination of communication networks. For example, communication network 654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 654 can be a local area network, a wide area network, a public network (e.g., the Internet), a private or semi-private network (e.g., a corporate or university intranet), any other suitable type of network, or any suitable combination of networks. Communications links shown in FIG. 6 can each be any suitable communications link or combination of communications links, such as wired links, fiber optic links, Wi-Fi links, Bluetooth links, cellular links, and so on.

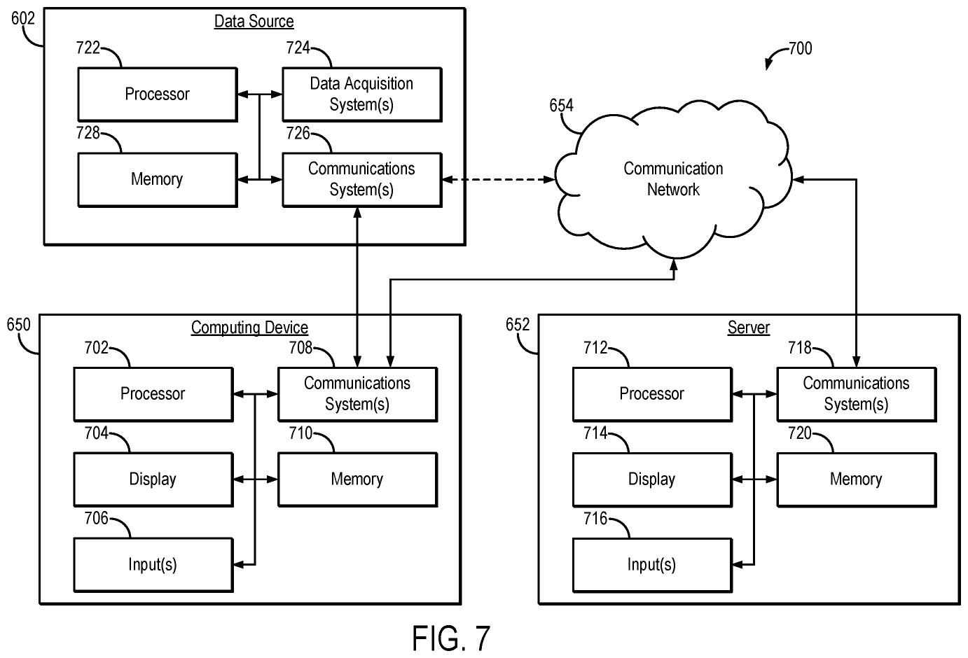

Referring now to FIG. 7, an example of hardware 700 that can be used to implement data source 602, computing device 650, and server 652 in accordance with some embodiments of the systems and methods described in the present disclosure is shown.

As shown in FIG. 7, in some embodiments, computing device 650 can include a processor 702, a display 704, one or more inputs 706, one or more communication systems 708, and/or memory 710. In some embodiments, processor 702 can be any suitable hardware processor or combination of processors, such as a central processing unit (“CPU”), a graphics processing unit (“GPU”), and so on. In some embodiments, display 704 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 706 can include any suitable input devices and/or sensors that can be used to receive user input, such as a keyboard, a mouse, a touchscreen, a microphone, and so on.

In some embodiments, communications systems 708 can include any suitable hardware, firmware, and/or software for communicating information over communication network 654 and/or any other suitable communication networks. For example, communications systems 708 can include one or more transceivers, one or more communication chips and/or chip sets, and so on. In a more particular example, communications systems 708 can include hardware, firmware, and/or software that can be used to establish a Wi-Fi connection, a Bluetooth connection, a cellular connection, an Ethernet connection, and so on.

In some embodiments, memory 710 can include any suitable storage device or devices that can be used to store instructions, values, data, or the like, that can be used, for example, by processor 702 to present content using display 704, to communicate with server 652 via communications system(s) 708, and so on. Memory 710 can include any suitable volatile memory, non-volatile memory, storage, or any suitable combination thereof. For example, memory 710 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 710 can have encoded thereon, or otherwise stored therein, a computer program for controlling operation of computing device 650. In such embodiments, processor 702 can execute at least a portion of the computer program to present content (e.g., images, user interfaces, graphics, tables), receive content from server 652, transmit information to server 652, and so on. For example, the processor 702 and the memory 710 can be configured to perform the methods described herein (e.g., the method 200 of FIG. 2, the method 300 of FIG. 3).

In some embodiments, server 652 can include a processor 712, a display 714, one or more inputs 716, one or more communications systems 718, and/or memory 720. In some embodiments, processor 712 can be any suitable hardware processor or combination of processors, such as a CPU, a GPU, and so on. In some embodiments, display 714 can include any suitable display devices, such as an LCD screen, LED display, OLED display, electrophoretic display, a computer monitor, a touchscreen, a television, and so on. In some embodiments, inputs 716 can include any suitable input devices and/or sensors that can be used to receive user input, such as a keyboard, a mouse, a touchscreen, a microphone, and so on.

In some embodiments, communications systems 718 can include any suitable hardware, firmware, and/or software for communicating information over communication network 654 and/or any other suitable communication networks. For example, communications systems 718 can include one or more transceivers, one or more communication chips and/or chip sets, and so on. In a more particular example, communications systems 718 can include hardware, firmware, and/or software that can be used to establish a Wi-Fi connection, a Bluetooth connection, a cellular connection, an Ethernet connection, and so on.

In some embodiments, memory 720 can include any suitable storage device or devices that can be used to store instructions, values, data, or the like, that can be used, for example, by processor 712 to present content using display 714, to communicate with one or more computing devices 650, and so on. Memory 720 can include any suitable volatile memory, non-volatile memory, storage, or any suitable combination thereof. For example, memory 720 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 720 can have encoded thereon a server program for controlling operation of server 652. In such embodiments, processor 712 can execute at least a portion of the server program to transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone), and so on.

In some embodiments, the server 652 is configured to perform the methods described in the present disclosure. For example, the processor 712 and memory 720 can be configured to perform the methods described herein (e.g., the method 200 of FIG. 2, the method 300 of FIG. 3).

In some embodiments, data source 602 can include a processor 722, one or more data acquisition systems 724, one or more communications systems 726, and/or memory 728. In some embodiments, processor 722 can be any suitable hardware processor or combination of processors, such as a CPU, a GPU, and so on. In some embodiments, the one or more data acquisition systems 724 are generally configured to acquire data, images, or both, and can include an MRI system. Additionally or alternatively, in some embodiments, the one or more data acquisition systems 724 can include any suitable hardware, firmware, and/or software for coupling to and/or controlling operations of an MRI system. In some embodiments, one or more portions of the data acquisition system(s) 724 can be removable and/or replaceable.

Note that, although not shown, data source 602 can include any suitable inputs and/or outputs. For example, data source 602 can include input devices and/or sensors that can be used to receive user input, such as a keyboard, a mouse, a touchscreen, a microphone, a trackpad, a trackball, and so on. As another example, data source 602 can include any suitable display devices, such as an LCD screen, an LED display, an OLED display, an electrophoretic display, a computer monitor, a touchscreen, a television, etc., one or more speakers, and so on.

In some embodiments, communications systems 726 can include any suitable hardware, firmware, and/or software for communicating information to computing device 650 (and, in some embodiments, over communication network 654 and/or any other suitable communication networks). For example, communications systems 726 can include one or more transceivers, one or more communication chips and/or chip sets, and so on. In a more particular example, communications systems 726 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 728 can include any suitable storage device or devices that can be used to store instructions, values, data, or the like, that can be used, for example, by processor 722 to control the one or more data acquisition systems 724, and/or receive data from the one or more data acquisition systems 724; to generate images from data; present content (e.g., data, images, a user interface) using a display; communicate with one or more computing devices 650; and so on. Memory 728 can include any suitable volatile memory, non-volatile memory, storage, or any suitable combination thereof. For example, memory 728 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 728 can have encoded thereon, or otherwise stored therein, a program for controlling operation of data source 602. In such embodiments, processor 722 can execute at least a portion of the program to generate images, transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

Various features and advantages of the invention are set forth in the following claims.

1. A method for breast tumor margin assessment, the method comprising:
administering a radiotracer containing a radioisotope of zinc to a patient;
positioning a positron detection probe proximate a region-of-interest in a breast of the patient;
acquiring positron emission data from the region-of-interest using the positron detection probe;
receiving the positron emission data with a computer system;
determining, using the computer system, a tumor margin in the region-of-interest from the positron emission data; and
generating a report indicating the determined tumor margin using the computer system.

2. The method of claim 1, wherein the radioisotope of zinc is ⁶³Zn.

3. The method of claim 2, wherein the radiotracer comprises ⁶³Zn-zinc citrate.

4. The method of claim 1, wherein the radiotracer is administered to the patient 20 to 30 minutes prior to acquiring the positron emission data.

5. The method of claim 1, wherein the positron detection probe is a handheld positron detection probe.

6. The method of claim 1, wherein the positron detection probe is coupled to a robotic arm of a surgical robot.

7. The method of claim 1, wherein positioning the positron detection probe proximate the region-of-interest comprises inserting the positron detection probe into a tumor resection cavity in the breast of the patient.

8. The method of claim 1, wherein the positron emission data indicates preferential uptake of the radiotracer in tissues within the region-of-interest.

9 .The method of claim 8, wherein determining the tumor margin comprises differentiating healthy tissue from tumor tissue by analyzing the positron emission data relative to a threshold level of radiotracer uptake.

10. The method of claim 9, wherein tissues having positron emissions above the threshold level are determined as part of the tumor margin.

11. The method of claim 1, wherein the report comprises a tumor margin map depicting a spatial distribution of the tumor margin as determined from the positron emission data.

12. The method of claim 11, wherein the tumor margin map includes pixels that are highlighted to represent tumor tissue in the determined tumor margin relative to healthy tissue not part of the tumor margin.

13. The method of claim 1, wherein the report is generated in real-time as the positron emission data are being acquired from the region-of-interest.

14. A method of breast tumor margin assessment, the method comprising the steps of:
receiving positron emission data with a computer system, wherein the positron emission data comprise measurements of positron emissions from breast tissues in a region-of-interest that have taken up a previously administered radiotracer containing a zinc radioisotope;
generating from the positron emission data, using the computing system, a tumor margin map that depicts a spatial distribution of the positron emissions in the region-of-interest; and
displaying the tumor margin map with the computer system.

15. The method of claim 14, wherein the positron emission data are received by the computer system from a positron detection probe.

16. The method of claim 14, wherein the tumor margin is determined based on a preferential uptake of the radiotracer by tissues in the region-of-interest.

17. The method of claim 16, wherein the tumor margin is determined based on comparing the measurement of positron emissions in the positron emission data with a threshold indicating the preferential uptake of the radiotracer.

18. The method of claim 14, wherein the positron emission data comprise measurements of radioisotope activity concentration.

19. The method of claim 14, wherein the radioisotope is ⁶³Zn.

20. The method of claim 19, wherein the radiotracer is ⁶³Zn-zinc citrate.
